(19) United States
(12) Patent Application Publication
(10) Pub. No.: US 2025/0257370 A1
(43) Pub. Date: Aug. 14, 2025

(54) SYNTHETIC ENZYME COMPLEXES FOR IN VITRO RUBBER PRODUCTION

(71) Applicant: The United States of America , as represented by the Secretary of Agriculture, Washington, DC (US)

(72) Inventors: GRISEL P. PONCIANO, Castro Valley, CA (US); COLLEEN M. MCMAHAN, Sausalito, CA (US); PAUL HOEPRICH, Sacramento, CA (US); PATRICK SCANNON, San Francisco, CA (US)

(21) Appl. No.: 19/079,787

(22) Filed: Mar. 14, 2025

Related U.S. Application Data
(63) Continuation of application No. 17/469,744, filed on Sep. 8, 2021, now Pat. No. 12,275,974.
(60) Provisional application No. 63/075,881, filed on Sep. 9, 2020.

(30) Foreign Application Priority Data
Sep. 9, 2021 (WO) PCT/US21/49605

(51) Int. Cl. C12P 5/02; C12N 9/10

(52) U.S. Cl. CPC C12P 5/026; C12N 9/1085; C12Y 205/0102

(57) ABSTRACT

Disclosed herein are recombinant enzymes derived from various rubber-producing plants and the use of those enzymes in the production of natural rubber outside of plant tissues. Systems and methods for the utilization of these enzymes in the production of natural rubber are also provided.

### CROSS-REFERENCE

The present application is a continuation of U.S. patent application Ser. No. 17/469,744 filed Sep. 8, 2021, which claims priority to U.S. Provisional Patent Application Ser. No. 63/075,881 filed on Sep. 9, 2020, the contents of which are expressly incorporated herein by reference in their entirety.

### BACKGROUND OF THE INVENTION

### Field of Invention

Disclosed herein are recombinant enzymes derived from various rubber-producing plants and the use of those enzymes in the production of rubber polymer outside of plant tissues. Systems and methods for the utilization of these enzymes in the production of rubber polymer are also provided.

### SEQUENCE LISTING

The instant application contains a Sequence Listing XML required by 37 C.F.R. § 1.831 (a) which has been submitted in XML file format via the USPTO patent electronic filing system and is hereby incorporated by reference in its entirety. The XML file was created on Mar. 14, 2025, is named Sequence_Listing_0045_25 and has 20.2 KB.

### Background

Natural rubber (NR) is a U.S. Critical Agricultural Material (Public Law 95-592), sourced mainly from Hevea brasiliensis (rubber tree), at over 13 M tons global production in 2019 (Association of Natural Rubber Producing Countries), and is imported to meet essential industry, medicine, and defense needs. Despite the availability of petroleum-based synthetic rubber, NR has outstanding chemical and physical properties that render it irreplaceable in applications such as aircraft tires and medical devices (Mooibroek and Cornish 2000). Global industrial growth suggests the demand of this raw material will eventually outstrip production capabilities. Production has not changed appreciably in generations, i.e. established plantation farms, located largely in Southeast Asia, still plant and grow trees that are manually tapped to collect crude rubber latex material. Despite being an irreplaceable raw material, no real advances affecting NR supply have occurred. As such, we have developed novel systems and methodologies for the in vitro production of rubber polymer utilizing synthetic enzyme complexes (SECs) comprised of recombinant enzymes, optionally supported and stabilized by natural or synthetic lipids and other stabilizing agents, into mimetic rubber synthesis devices.

### SUMMARY OF THE INVENTION

The present disclosure provides, in one embodiment, compositions for producing rubber polymer in vitro, comprising: 1) an isolated and purified cis-prenyltransferase; 2) an isolated and purified cis-prenyltransferase binding protein; 3) a reaction initiator; and 4) a substrate convertible into a polymer by said cis-prenyltransferase. In some embodiments, the compositions also comprise a lipid capable of forming a higher order structure, such as a micelle, a liposome, a mono-layer membrane, or a bilayer membrane. In specific embodiments, the lipid is DMPC. In additional embodiments, the compositions comprise an isolated and purified small rubber particle protein, such as one that is at least 99% identical to SEQ ID NO: 10 or SEQ ID NO: 12. In particular embodiments, the cis-prenyltransferase has a sequence at least 99% identical to SEQ ID NO: 2 or SEQ ID NO: 6. In some embodiments, the cis-prenyltransferase binding protein has a sequence at least 99% identical to SEQ ID NO: 4 or SEQ ID NO: 8. In some embodiments, the compositions also comprises a non-aqueous organic solvent. In still other embodiments, the compositions comprise a divalent cation, such as Mg²⁺. In some embodiments, the initiator is an allylic pyrophosphate, such as FPP.

The present disclosure provides in another embodiment, methods of synthesizing rubber polymer, comprising the steps of: 1) providing reagents for synthesizing the rubber polymer; 2) providing the composition having a) an isolated and purified cis-prenyltransferase; b) an isolated and purified cis-prenyltransferase binding protein; c) a reaction initiator; and d) a substrate convertible into a polymer by said cis-prenyltransferase; and 3) contacting the reagents with the composition of claim 1 under conditions allowing for the production of the rubber polymer, thereby synthesizing the rubber polymer. In some embodiments of this method, the composition of step 2 further comprises a lipid capable of forming a higher order structure. In other embodiments of the methods, the composition of step 2 also contains an isolated and purified small rubber particle protein. In still other embodiments of the methods, the composition of step 2 also comprises an organic solvent.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides a pictorial representation of one potential embodiment of a system for producing rubber polymer ex vivo.

FIG. 2 provides graphic representation of the results of an 14C-IPP incorporation assay of recombinant CPT and CBP under two different environmental conditions, aqueous (aq) and aqueous-organic biphasic (bi).

FIG. 3 provides a gel permeation chromatogram tracing from a refractive index detector showing reaction products of an in vitro rubber polymer synthesis reaction.

FIG. 4 provides a gel permeation chromatogram tracing from a light scattering detector showing reaction products of an in vitro rubber polymer synthesis reaction.

FIG. 5 provides graphical representation of data from in vitro polymerization using Hevea recombinant proteins HRT2 (Hevea Rubber Transferase 2; homologue to guayule CPT) and HRBP (Hevea Rubber Transferase Binding Protein; homologue to guayule CBP).

Experimental=recombinant proteins; Negative control=recombinant proteins+40 mM EDTA

FIG. 6 provides results from a chromatogram showing elution for THF-soluble reaction components. Polyisoprene molecular weight standards are represented by 1PI1 (1030 g/mole), 3PI3 (12,100 g/mole), 5PI5 (111,000 g/mole) and 7PI7 (1,180,000 g/mole). Reaction product replicates are represented by 2GP1-2, 4GP3-4, and 5GP5-6.

FIG. 7 provides results from a chromatogram showing elution for THF-soluble reaction components, zoomed out, to show the height of the peaks. Polyisoprene molecular weight standards are represented by 1PI1 (1030 g/mole), 3PI3 (12,100 g/mole), 5PI5 (111,000 g/mole) and 7PI7 (1,180,000 g/mole). Reaction product replicates are represented by 2GP1-2, 4GP3-4, and 5GP5-6.

### DETAILED DESCRIPTION OF THE INVENTION

Disclosed herein are rubber-polymer-producing synthetic molecular complexes. These synthetic complexes may be comprised of several forms, e.g., 1) lipid/protein monolayer spherical particles (as found in nature), 2) lipid/protein bilayer sheets, 3) lipid/protein single layer sheets/films, 4) micelles, 5) liposomes, 6) nanolipoprotein particles (NLP), vesicles used to stabilize membrane-bound enzymes, and 7) molecular complexes bound to solid surfaces including beads as support structures. Particular embodiments of the present subject invention utilize a minimally required enzyme component, namely: 1) a cis-prenyl transferase (CPT), and 2) a cis-prenyl transferase binding (CBP) protein. Proteins utilized are typically produced by purification from recombinant microbes. In preferred embodiments, the two enzymes are incorporated into a membrane composed of lipids. Rubber-polymer-producing systems disclosed herein can also contain additional components for stabilization of the system, such as a Small Rubber Particle Protein (SRPP), a Rubber Elongation Factor (REF), or synthetic apolipoproteins (e.g., recombinant Apo-A1, human cat #SRP6410, Sigma-Aldrich). Other known rubber particle proteins such as allene oxide synthase (AOS), can also be utilized in the rubber-polymer-producing systems and methods disclosed herein. The systems, methods, and compositions provided herein lack certain components of plant-derived natural rubber particles, including, but not limited to, ATP synthase subunits and transporter-like proteins.

Rubber polymers of the present disclosure are like natural rubber, for example have high molecular weight and high stereospecificity, but the ex vivo material is likely to be substantially purer than plant derived natural rubber. Additionally, molecular weight distribution might be different between ex vivo rubber polymers and NR. Ex vivo rubber polymer can also be made utilizing different initiator molecules, thus potentially having an overall different polymer structure at the end group. Such initiators can potentially contain reactive moieties. Systems described herein can also be fed alternative monomers, which are also polymerizable by the synthetic complex to create new synthetic polymers with high stereospecificity; can be fed a mixture of monomers to create random and block copolymers or branched polymers; and can be fed at the end of a reaction to “cap” the polymer, resulting in a chain-end functionalized polymer. The rubber-polymer-producing systems (i.e., an engineered rubber particle) can be used to produce ex vivo (plant-free) rubber polymer from bio-derived monomers (e.g. IPP, bioisoprene), for example using cellulosic sugar and the like as a starting material. The subject synthetic technology can

be used to complement current rubber production, particularly for specialized applications such as medical supplies, due to the likelihood of obtaining a pure product lacking in allergenic proteins, or micro/nano systems inaccessible with current technology. Further, it provides an additional layer of supply security and enables rubber polymer production in extreme environments such as deep ocean and outer space.

Constructs containing the engineered rubber particles described herein can be housed in a complex that enables application of industrial biotechnology production/manufacturing tenets to ex vivo production, allowing for the development of scalable processes leading to lower overall raw material and production costs.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state. One non-limiting example of an isolated substance is a recombinant protein purified from a suitable host microbe producing the protein.

The term “about” is defined as plus or minus ten percent of a recited value. For example, about 1.0 g means 0.9 g to 1.1 g and all values within that range, whether specifically stated or not.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The terms “polypeptide, peptide or protein” refer to polymers in which the monomers are amino acid residues which are linked through amide bonds. When the amino acids are alpha-amino acids, either the L-optical isomer or the D-optical isomer can be used. The terms are used interchangeably herein. These terms apply to amino acid polymers in which one or more amino acid residues are an artificial chemical mimetic of a corresponding naturally occurring amino acid, as well as to naturally occurring amino acid polymers and non-naturally occurring amino acid polymers.

The term “recombinant” when used with reference, e.g., to a cell, or nucleic acid, protein, or vector, indicates that the cell, organism, nucleic acid, protein or vector, has been modified by the introduction of a heterologous nucleic acid or protein or the alteration of a native nucleic acid or protein, or that the cell is derived from a cell so modified. Thus, for example, recombinant cells may express genes/polynucleotides that are not found within the native (non-recombinant or wild-type) form of the cell or express native genes in an otherwise abnormal amount-over-expressed, under-expressed or not expressed at all-compared to the non-recombinant or wild-type cell or organism.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch, J Mol Biol, (1970) 48:3, 443-53). A computer-assisted sequence alignment can be conveniently performed using a standard software program such as GAP which is part of the Wisconsin Package Version 10.1 (Genetics Computer Group, Madison, Wisconsin, USA) using the default scoring matrix with a gap creation penalty of 50 and a gap extension penalty of 3.

The phrase “high percent identical” or “high percent identity”, and grammatical variations thereof in the context of two polynucleotides or polypeptides, refers to two or more sequences or sub-sequences that have at least about 80%, identity, at least about 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid identity, when compared and aligned for maximum correspondence, as measured using a sequence comparison algorithm or by visual inspection. In an exemplary embodiment, a high percent identity exists over a region of the sequences that is at least about 16 nucleotides or amino acids in length. In another exemplary embodiment, a high percent identity exists over a region of the sequences that is at least about 50 nucleotides or amino acids in length. In still another exemplary embodiment, a high percent identity exists over a region of the sequences that is at least about 100 nucleotides or amino acids or more in length. In one exemplary embodiment, the sequences are high percent identical over the entire length of the polynucleotide or polypeptide sequences.

### Proteins

Proteins utilized in the methods, systems and processes provided herein typically derive from natural sources, but also include modified versions of such proteins. Proteins can be enzymes, such as a cis-prenyltransferase or a cis-prenyltransferase binding protein from any source, including an NR-producing plant. Proteins can also be a trans-prenyl transferase or trans-prenyl transferase binding protein from any source, including a trans-NR (Gutta percha) producing plant. Proteins can also be non-enzymatic proteins such as Small Rubber Particle Protein (SRPP), which is thought to provide structural support to enzymes involved in NR production. Typically, because of the quantities of proteins desired for large-scale in vitro production of NR, proteins are produced recombinantly, and not isolated from naturally occurring sources. However utilized, proteins of the present invention are not incorporated into a plant rubber particle, where rubber synthesis is known to occur in plants and in enzymatically active rubber particles extracted from plants.

Any bacterial or eukaryotic recombinant protein production system can be utilized in practicing the instant disclosure, including, but not limited to E. coli, yeasts, filamentous fungi, insect cells, and mammalian cells. In the construction of a recombinant protein production system, it may be necessary or desirable to modify a source DNA coding region to a codon-optimized version in order to achieve desired expression and production levels. Such modifications are well within the capabilities of one skilled in the art. If desired, cell-free systems can also be utilized for protein production.

Table 1 provides a brief list of exemplary proteins that can be utilized in practicing the methods, systems and compositions provided herein. The listed proteins are derived from two NR-producing plants (guayule and Hevea rubber tree), however, orthologs from other species are known and can be utilized, including from other known NR-producing plants (e.g., Russian dandelion (Taraxacum kok-saghyz), lettuce (Lactuca sativa), rubber fig tree (Ficus elastica), Eucommia ulmoides, and Castilla elastica, other plants, and non-plant species (e.g., Lactarius volemus, Lactarius chrysorrheus, and Lactarius hygrophoroides). In the case of transferase proteins based on Palaquia gutta, a trans-polyisoprene polymer would be produced.

TABLE 1
Exemplary Recombinant Proteins

| Enzyme Name | Original Source | SEQ ID NO: |
|---|---|---|
| cis-prenyltransferase | Partheniuum argentatum | SEQ ID NO: 2 |
| (CPT) | (guayule) | |
| cis-prenyltransferase | P. argentatum | SEQ ID NO: 4 |
| binding protein (CBP) | | |
| cis-prenyltransferase | Hevea brasiliensis | SEQ ID NO: 6 |
| (HRT2) | (rubber tree) | |
| cis-prenyltransferase | H. brasiliensis | SEQ ID NO: 8 |
| binding | | |
| protein (HRBP) | | |
| Small rubber | P. argentatum | SEQ ID NO: 10 |
| particle protein | | |
| (PaSRPP) | | |
| Small rubber | H. brasiliensis | SEQ ID NO: 12 |
| particle protein | | |
| (HbSRPP) | | |
| Rubber Elongation | H. brasiliensis | SEQ ID NO: 14 |
| Factor (REF) | | |

### Reaction Parameters

Reactions described herein can be modified by the skilled artisan to achieve efficiency or other desired outcomes. Such modifications are known in the art. Exemplary modifications include reaction pH between 6.5 and 9.5, including any and all specific pH values between these endpoints, such as 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, and 9.5. Additionally, reaction temperature is an exemplary modification, such as temperatures at 10° C.-35° C., including any and all specific values between these two endpoints, such as 10° C., 10.5° C., 11° C., 11.5° C., 12° C., 12.5° C., 13° C., 13.5° C., 14° C., 14.5° C., 15° C., 15.5° C., 16° C., 16.5° C., 17° C., 17.5° C., 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C. Another exemplary modification is alteration of the buffer system, such as Tris-HCl buffers at concentrations between 10-150 mM, including any and all specific concentrations between these two endpoints, such as 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, and 150 mM. Additional exemplary modifications include the presence or absence of reaction enhancers and surface area, such as cobalt resin present in the reaction and/or the presence of beads or matrices.

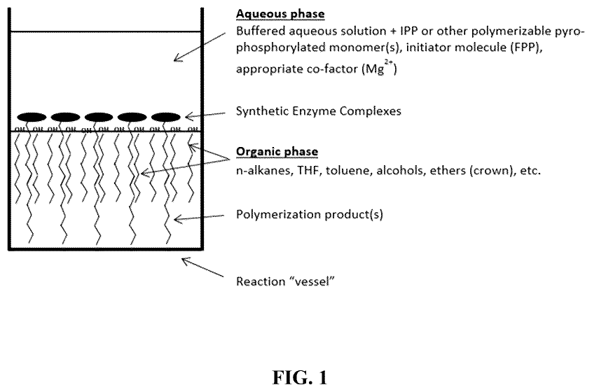

Proteins of the present invention can be combined with multiple additional components as desired for fine-tuning of ex vivo rubber-polymer-producing reactions. One non-limiting, exemplary embodiment is represented in FIG. 1, which shows various optional components of systems utilizing disclosed proteins, enzymes and combinations thereof. Polymerization rates and efficiencies are expected to be impacted, and therefore potentially optimized, by judicious selection of 1) pH (pH 6.5-9.5), 2) magnesium or other divalent cation cofactor choice and concentration (1-20 mM), 3) lipid choice (saturated, unsaturated, furan structures), 4) lipid concentration (0.5-5%), 5) temperature (10° C. to 35° C.), and 6) presence or absence of one or more solvents.

### Lipids

Lipids useful in practicing the instant invention can be natural or synthetic, typically possessing both hydrophilic and hydrophobic properties that in an aqueous environment assemble in a lipid monolayer or bilayer structure. Such polar lipids have a hydrophilic moiety and a hydrophobic moiety, as exemplified by phospholipids (e.g., PE, PC, PI, PS, PA, DMPC, DOPE, DOPC, DPPC), glycolipids (e.g., glycosphingolipids), sterols (e.g., sitosterol), sphingolipids (e.g., ceramides), neutral lipids (e.g., triglycerides, ether lipids, furanoid lipids, lipids with hydroxy fatty acids), and alkylphosphocholines. Specific lipids that can be utilized include, but are not limited to Phosphatidylethanolamine (PE), phosphatidylcholine (PC), phosphatidylinositol (PI), phosphatidylserine (PS), phosphatidic acid (PA), dimyristoylphosphatidylcholine (DMPC), dioleoylphosphoethanolamine (DOPE), dioleoylphosphatidylcholine (DOPC), and dipalmitoylphosphatidylcholine (DPPC). Additional lipids are widely known in the art and can be selected by the skilled artisan as suitable for particular embodiments (e.g., high temperature reactions, low temperature reactions, high pH reactions, etc.).

Reaction initiators that can be utilized in practicing the instant disclosure include any now known, or discovered in the future. Non-limiting exemplars of initiators include allylic pyrophosphates such as FPP, di-methyl allyl PP (DMAPP), geranyl PP (GPP), geranyl geranyl PP (GGPP), HPP, derivatives of allylic pyrophosphates (e.g., benzophenone), and FOO. For embodiments utilizing an organic compound for a bi-phasic system, an appropriate organic compound (e.g., organic solvent) can be chosen by the skilled artisan. Exemplary compounds include benzene, n-hexane, cyclohexane, tetrahydrofuran, chloroform, and octanol. Alternative monomers/initiators

The native rubber transferase complex is known to accept a wide range of initiators; when such initiators are introduced to purified washed rubber particles IPP is polymerized. Alternative initiators include dimethyl allyl pyrophosphate (DMAPP), geranyl pyrophosphate (GPP), geranyl geranyl pyrophosphate (GGPP), hexa-hepta prenyl pyrophosphate (H-HPP) and others (Mau and Cornish U.S. Pat. No. 8,013,213; Mau et al. 2003). Even initiator analogs such as benzophenone derivatives of DMAPP and GPP successfully initiate polymerization despite the presence of the sizable R group (Xie et al. 2008). A similar range of alternative initiators may be used with the subject synthetic complexes, resulting in polymers with a functionalized (alpha) end group. In an analogous manner, a variety of pryophosphorylated molecules can serve as alternative monomers in the subject synthetic complexes to synthesize a variety of previously undescribed polymeric materials. For example, polymerization of (E-1)-hydroxy-2-methyl-2-butenyl-4-pyrophosphate (HMBPP) by the synthetic complex results in a hydroxy functionalized polyisoprene. Based on the structure of IPP, the native monomer, one group of alternative monomers that can be utilized in practicing the instant invention are derivatives of IPP with substitution at the R5, methyl group position, per Formula I. Non-limiting examples of such IPP derivatives include hydroxy IPP (R5=hydroxy group), aniline IPP (R5=aniline), and styrene IPP (R5=styrene).

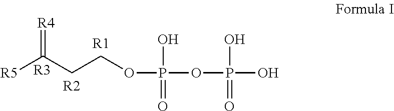

Having generally described this invention, the same will be better understood by reference to certain specific examples, which are included herein to further illustrate the invention and are not intended to limit the scope of the invention as defined by the claims.

### EXAMPLES

### Example 1

### Production and Isolation of Recombinant Proteins

Proteins CPT (SEQ ID NO: 2), CBP (SEQ ID NO: 4), and PaSRPP (SEQ ID NO: 10) were produced in E. coli, extracted by chemical lysis, and purified by affinity chromatography. Every step of the process was monitored by denaturing polyacrylamide gel electrophoresis (PAGE) and western blot analysis.

These proteins derived from guayule (Table 1) were cloned into pET29a (+) (NOVAGEN) protein expression vector and transformed into T7 Express (New England Biolabs, Ipswich, MA) E. coli cells. Bacterial cultures were grown at 37° C. in 50 mL Luria Bertani broth supplemented with 30 μg/mL of Kanamycin under moderate shaking until reaching a growth of optical density between 0.3-0.4 at A600. Protein synthesis was elicited with a final concentration of 0.4 mM IPTG for 30 min. Bacterial proteins were extracted with B-PER bacterial protein extraction reagent supplemented with DNasel, lysozyme, and proteinase cocktail (Pierce Biotechnology, Rockford IL). Recombinant proteins were affinity purified by bulk with S-protein agarose following manufacturer recommendations (Sigma-Millipore, USA), concentrated with Amicon 10K filters (Amicon, USA), and buffer exchanged with ZEBAspin columns (Pierce Biotechnology, Rockford IL) to replace the buffer with IPP incorporation assay buffer. Total protein concentration was estimated with QUICK START Bradford Protein Assay (Bio-Rad, Hercules, CA).

After normalizing protein concentrations, proteins were run on a 4-12% NUPAGE Bis-Tris pre-cast polyacrylamide gel under reducing conditions (Life Technologies, Carlsbad, CA) and detected with Bio-Safe Coomassie stain (Bio-Rad, Hercules, CA). For western blot, proteins from the PAGE gel were transferred to a PVDF membrane, blocked for 1 hr at room temperature or overnight at 4° C. with SUPERBLOCK (Pierce Biotechnology, Rockford IL) blocking buffer, incubated for 1 hr with 10 ng/ml anti S-Tag monoclonal antibodies (EMD Millipore, Temecula, CA), following 30 min incubation with 2 ng/ml anti Mouse IgG-HRP conjugate (Bio-Rad, Hercules, CA), and final chemiluminescent detection with SUPERSIGNAL Femto Maximum Sensitivity Substrate (Thermo Scientific, Waltham, MA). Membrane was wrapped with plastic wrap and exposed to CL-XPOSURE film (Thermo Scientific, Waltham, MA) for 10-30 s. Protein molecular weight markers are PAGERULER prestained protein ladder (Invitrogen, USA).

HRT2 (SEQ ID NO: 6), HRBP (SEQ ID NO: 8), and HbSRPP (SEQ ID NO: 12) proteins derived from the rubber tree (Table 1) were cloned into pET (NOVAGEN) protein expression vector pET32b (+) and transformed into E. coli cells (BL21 (DE3) (New England Biolabs, Ipswich, MA). Bacterial cultures were grown at 37° C. in 50 mL Luria Bertani broth supplemented with 50 μg/mL carbenicillin under moderate shaking until reaching a growth of appropriate optical density at A600 (0.6). Protein synthesis was elicited with a final concentration of 0.4 mM IPTG for 2-6h. Bacterial proteins were extracted by chemical lysis using CELLYTIC (Sigma-Millipore, USA). Recombinant proteins were affinity purified (by column fractionation with Cobalt Resin (Thermo Fisher, USA). When needed, purified proteins were concentrated with Amicon 10K filters (Amicon, USA) or buffer exchanged with ZEBA spin columns (Pierce Biotechnology, Rockford IL) to replace the buffer with IPP incorporation assay buffer.

Every step of the protein extraction and purification process was monitored by standard denaturing polyacrylamide gel electrophoresis (PAGE) and western blot analysis. For western blot, proteins from the PAGE gel were transferred to a PVDF membrane, blocked for 1 hr at room temperature or overnight at 4° C. with SUPERBLOCK (Pierce Biotechnology, Rockford IL) blocking buffer. The membrane was incubated with anti HIS-tag primary antibody overnight at 4° C., then with horse radish peroxidase-secondary antibody at room temperature for 1h prior to colorimetric development with CN/DAB substrate kit (Thermo Fisher, USA).

### Example 2

### Assembly of Enzyme Complex and Polymerization

Recombinant proteins CPT (SEQ ID NO: 2) and CBP (SEQ ID NO: 4) (250 ng-14 μg) were incubated with an equal volume of 12 mM DMPC (1,2-Dimyristoyl-sn-glycero-3-phosphocholine) and 20 mM cholate for Ihr at room temperature. A 20 μL aliquot of the self-assembled protein/lipid complex was transferred to a centrifugal filter unit (MilliporeSigma Model Ultrafree® MC-VV) containing 20 μM farnesyl pyrophosphate (FPP) initiator, 1 mM unlabeled IPP and 0.9 nmol (55 mCi mmol-1) 14C-IPP in buffer (100 mM Tris-HCl, pH 7.5; 1.25 mM MgSO4, 5 mM dithiothreitol) in a total reaction volume of 50 μL. Under these conditions, the initiator molecule farnesyl pyrophosphate (FPP) binds to the rubber transferase enzyme complex, and the polymerization, proceeds by successive condensation reactions of the monomer-isopentenyl pyrophosphate (IPP, both 14C-labelled and un-labelled).

The reactions, performed in triplicate, were incubated at room temperature for 3-4 h, stopped with addition of 40 mM ethylene diaminetetraacetic acid (EDTA), pH 8.0, and washed by centrifugation at 14,000 rpm three times with water for removal of unincorporated monomer (IPP). For the biphasic condition, the same reaction was conducted and 25 μL of either butanol or hexane was carefully layered on top of the reaction. For product molecular weight analysis, the same reaction was conducted without radioactive 14C-IPP monomer.

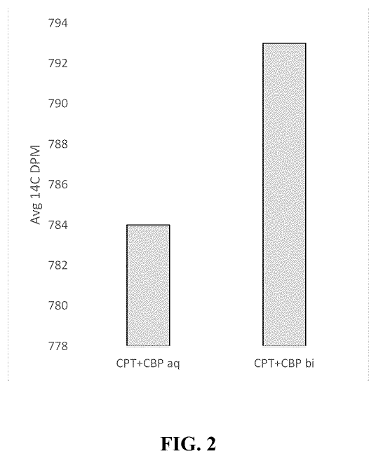

Following the IPP/14C IPP polymerization, the filter unit containing the washed product was inserted in a vial containing 2 mL BD Cocktail (Fisher Sci., Model ScintiVerse, Santa Clara, CA) and the amount of [14C]-IPP in each individual filter was quantified by scintillation counting (Beckman Coulter, Model LS 6500, Brea, CA). The average value of the 14C disintegrations per minute (DPM) count quantifies the cis-prenyl transferase activity of the reaction mixture, as has been well established in native systems (Xie et al, Phytochem. (2008) 69:2539-45; Brasher et al., The Plant J. (2015) 82:903-14; Qu et al, J. Biol. Chem., (2015) 290:1898-1914; Placido et al, Front. Plant Sci. (2019) doi.org/10.3389/fpls.2019.00760). Results are shown in FIG. 2 for experiments utilizing CPT (SEQ ID NO: 2) and CBP (SEQ ID NO: 4) derived from guayule.

As stated above, the average value of the 14C disintegrations per minute (DPM) count from the polymers produced quantifies the cis-prenyl transferase enzymatic activity. The DPM values for a series of experiments wherein initiator and monomer molecules were introduced into reaction vessels containing various enzymatic and stabilizing components are shown below. The higher the value, the more IPP incorporated into the product under the specific conditions and times. CPT (SEQ ID NO: 2) enzyme and CBP (SEQ ID NO: 4) were expressed in E. coli and affinity purified. Activity of the proteins were tested by mixing them in equal amounts on a microfuge tube in the presence of the synthetic phospholipid 1,2-Dimyristoyl-sn-glycero-3-phosphocholine (DMPC) to stabilize these two membrane-associated proteins.

The 14C-IPP incorporation assay is an in vitro enzymatic assay to test the activity of CPTs. In this assay, the molecule farnesyl pyrophosphate (FPP) binds to the CPT enzyme to initiate the reaction, followed by successive condensation reactions of isopentenyl pyrophosphate (IPP, both 14C-labelled and un-labelled) that synthesizes an isoprene polymer of varying length. After 3-4 hrs of incubation at room temperature with gentle shaking, the reaction is stopped, and unpolymerized FPP and IPP monomers removed from the reaction product. To evaluate 14C-IPP incorporation into the final isoprene polymer, the reaction is mixed with scintillation fluid and the radiation emitted by the incorporated 14C-IPP is detected on a scintillation counter in the form of DPM (disintegrations per minute) units.

We conducted the 14C-IPP incorporation assay under two different environmental conditions: aqueous (aq) and aqueous-organic bi-phasic (bi). The reason for testing these two conditions is the isoprene polymer is hydrophobic in nature and the aqueous environment is predicted to be a suboptimal one for effective in vitro synthesis. The addition of an organic solvent layer (such as n-hexane or butanol) on top of the aqueous reaction can provide an environment where the polymer can easily be solubilized without hindering the reaction.

Similar experiments were run utilizing HRT2 (SEQ ID NO: 6) and HRBP (SEQ ID NO: 8) either alone or in combination with SRPP (SEQ ID NO: 10) and an NLP (made from 5 mg DMPC and 1 mg SRPP). Pre-incubation was performed for 1 hour at room temperature. Results are shown in Table 2 for experiments utilizing HRT2 (SEQ ID NO: 6) and HRBP (SEQ ID NO: 8).

TABLE 2
IPP incorporation results utilizing rubber tree derived proteins

| Reaction components and conditions | Average DPM |
|---|---|
| HRT2 + HRBP, biphasic | 346 |
| HRT2 + HRBP + DMPC, biphasic | 4024 |
| HRT2 + HRBP + SRPP − NLP | 55 |
| (no pre-incubation), aqueous | |
| HRT2 + HRBP + SRPP − NLP | 258 |
| (pre-incubation), biphasic | |

### Example 3

### Characterization of Reaction Products

Gel permeation chromatography (GPC) i.e. liquid chromatography with continuous tetrahydrofuran (THF) solvent phase, using size exclusion separation columns and multiple detectors, was used to determine the quantity and molecular weight of the products of various in vitro reactions.

Reaction Products from Synthetic Complex HRT2+HRBP, Biphasic Reaction

Triplicate unfiltered reaction mixtures were combined for a total 150 μL volume, to which 300 μL THF was added. The mixture was gently shaken, for 12-18 hours solubilization time, then a100 μL aliquot was withdrawn from the (upper) organic phase, placed into an autosampler vial insert, and 50 μL load injected onto the columns and size exclusion separated (THF continuous phase, 1.0 mL/min) by two Agilent PL gel 10 μm Mixed-B columns in series at 35° C. Peak elution detected by refractive index (RID; Agilent 1260 Infinity, dn/dc=0.129) and light scattering (LS) (DAWN Helcos-II, Wyatt Technology Corp., Santa Barbara, CA) detectors and represents the polyisoprene. The molecular weights were calculated from the RID and LS peaks by Astra 6.1 (Wyatt Technology).

### Product Mass and Molecular Weight

Plant dolichols and polyprenols have variable molecular weights. Jankowski et al, (Plant Physiol. (1994) 143:448-452) report up to 25 isoprene units, or 125 carbon atoms, from various plant sources. Brasher et al. (supra) report up to 19 units, or about 90 carbon atoms. These correspond to molecular weight size in the 1500-2000 Da range. We have produced much longer chain polyisoprenes utilizing recombinant enzymes.

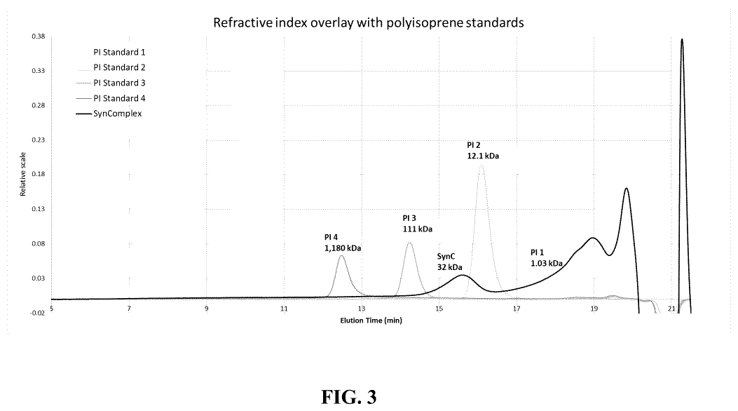

In the example HRT2+HRBP, biphasic experiment, recombinant proteins HRT (SEQ ID NO: 6) and HRBP (SEQ ID NO: 8) were prepared and combined with DMPC lipid to form complexes that polymerized and added cold IPP monomer with hexane as the organic phase. The molecular weight of the product was determined. Results confirm the presence of polyisoprene of ˜32,000 Da (Mp), ranging from below 10,000 to 100,000 Da (SynC peak below). Integration of the peak at yielded 6.77 μg of product in this molecular weight range. Lower molecular weight product was also produced, as evident by peaks at and above 19 minutes elution time. Negative peaks between 20-24 minutes are an artifact of sample injection. Chromatograms shown in FIG. 3 are from the Refractive index detector, set to dn/dc of 0.129, which corresponds to the known polyisoprene value (Jackson et al, J Appl. Polymer Sci. (1996), 61:865-874).

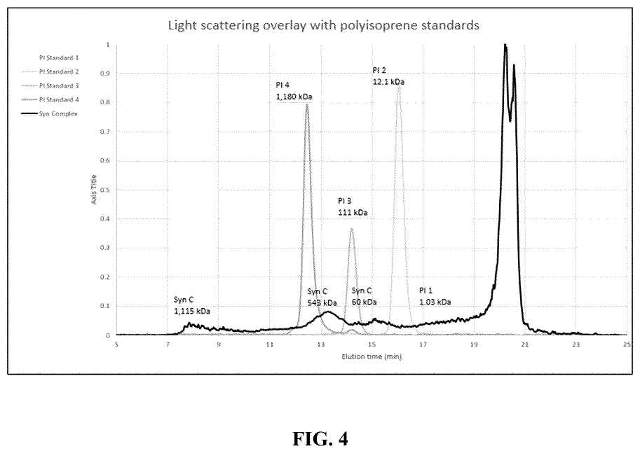

Results also suggest the presence of a very small amount of a very high molecular weight product, Mp of about 1,115,000 Da (Mp). Chromatograms shown in FIG. 4 are from the light scattering detector. Due to the very low amount of material, the quantity could not be determined. It is known in the art that light scattering detectors are more sensitive in the high molecular weight range, while refractive index detectors are more sensitive in the lower molecular weight range (Jeng et al, Appl. Polymer Sci. (1993) 49:1359-1374).

### Example 4

### Assembly of Enzyme Complex and Polymerization II

HRT2 (SEQ ID NO: 6) and HRBP (SEQ ID NO: 8) were expressed in E. coli and affinity purified. Activity of the proteins were tested by mixing them in equal amounts on a microfuge tube in the presence of the soy phosphatidylcholine (PC) to stabilize these two membrane-associated proteins. Recombinant proteins (15 μg) were mixed with 1% (w/v) soy phosphatidylcholine (PC) and protein/lipid complex was transferred to a centrifugal filter unit (MilliporeSigma Model Ultrafree® MC-VV) containing 5 μM farnesyl pyrophosphate (FPP) initiator, 1 mM unlabeled IPP and 0.9 nmol (55 mCi mmol-1) 14C-IPP in buffer (100 mM Tris-HCl, pH 8; 1.25 mM MgSO4, 20 mM 2-mercaptoethanol) in a total reaction volume of 100 μL. Negative control reactions included the same components plus EDTA to 40 mM final concentration.

The reactions, performed in triplicate, were incubated at room temperature for ˜18 h, stopped with addition of 40 mM ethylene diaminetetraacetic acid (EDTA), pH 8.0, and washed by centrifugation at 14,000 rpm four times with water for removal of unincorporated monomer (IPP). Following the IPP/14C-IPP polymerization, the filter unit containing the washed product was inserted in a vial containing 2 mL BD Cocktail (Fisher Sci., Model ScintiVerse, Santa Clara, CA) and the amount of 14C-IPP in each individual filter was quantified by scintillation counting (Beckman Coulter, Model LS 6500, Brea, CA). The average value of the 14C disintegrations per minute (DPM) count quantifies the cis-prenyl transferase activity of the reaction mixture, as has been well established in native systems (Xie et al, Phytochem. (2008) 69:2539-45; Brasher et al., The Plant J. (2015) 82:903-14; Qu et al, J. Biol. Chem., (2015) 290:1898-1914; Placido et al, Front. Plant Sci. (2019) doi.org/10.3389/fpls.2019.00760).

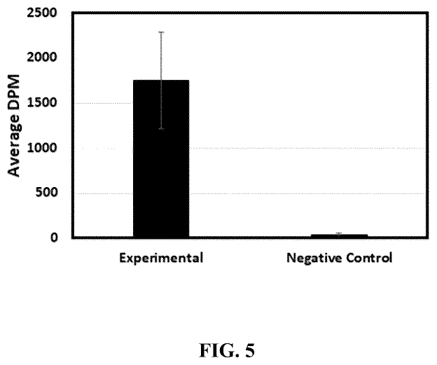

Results are shown in FIG. 5 for experiments utilizing HRT2 (SEQ ID NO: 6) and HRBP (SEQ ID NO: 8) derived from Hevea. As stated above, the average value of the 14C disintegrations per minute (DPM) count from the polymers produced quantifies the cis-prenyl transferase enzymatic activity. The DPM values for a series of experiments wherein initiator and monomer molecules were introduced into reaction vessels containing various enzymatic and stabilizing components are shown below. The higher the value, the more IPP incorporated into the product under the specific conditions and times.

### Characterization of Synthetic Enzyme Complex Reaction Products

Reaction products from this system were evaluated after 23h incubation. Six reaction vials, each 100 μL, were combined as: 1+2, 3+4, 5+6. 300 μL THF added, gentle shaking at room temperature, 4.5h. Four synthetic polyisoprene standards were prepared by placing pre-weighed polymer in 3 mL THF each, also shaken. GP1-2, GP3-4, GP5-6 solutions (100 μL, water clear) were pipetted into autosampler vials. Solution was notably viscous. All samples were injected (50 μL load) into the GPC, and chromatograms were collected per standard laboratory procedure.

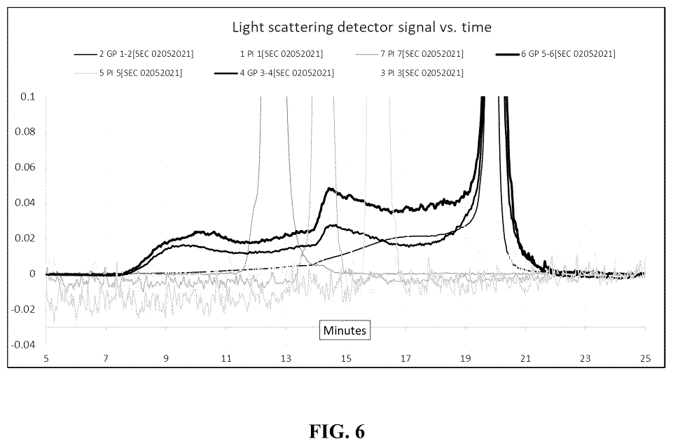

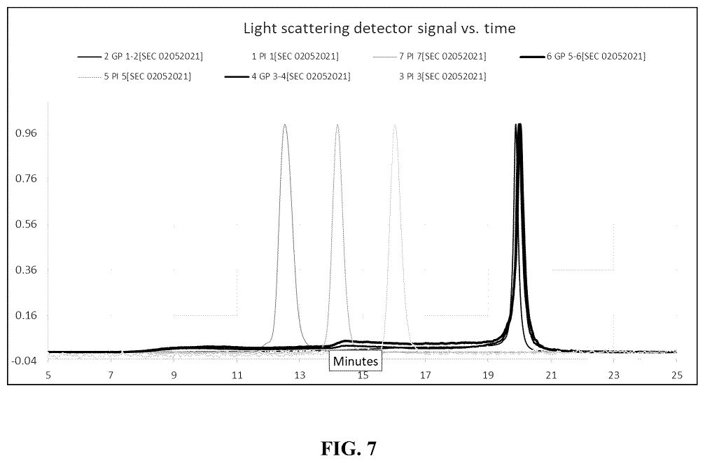

Results are shown in FIG. 6 and FIG. 7. Chromatograms from size-exclusion chromatographic (SEC) separation, which shows the elution of reaction products and synthetic polyisoprene standards. In SEC the larger molecules elute first. The polyisoprene standards serve as a calibration of elution time and molecular weight; elution from ˜12 to ˜19 minutes corresponded to 1,180,000 to 1030 g/mole. Reaction products from 3 replicate synthetic complex experiments (1+2, 3+4, 5+6) are also shown, and elute from ˜8-22 minutes. Similar elution profiles are seen for samples 3+4 and 5+6; sample 1+2 shows a different profile with elution of product beginning ˜14 minutes. For all 3 samples, the largest peak occurred at ˜20 minutes, interpreted as molecular weights below 1030 g/mole (see inset). Nevertheless, a small but measurable amount of high molecular weight product was detected, in all cases, at ˜105 g/mole. Reaction products (samples 3+4 and 5+6) that elute before 11 minutes represent products larger than 106 g/mole.

While the invention has been described with reference to details of the illustrated embodiments, these details are not intended to limit the scope of the invention as defined in the appended claims. The embodiment of the invention in which exclusive property or privilege is claimed is defined as follows:

We claim:

1. A composition for producing rubber polymer in vitro, comprising:
a. an isolated and purified cis-prenyltransferase;
b. an isolated and purified cis-prenyltransferase binding protein;
c. a reaction initiator; and
d. a substrate convertible into a polymer by said cis-prenyltransferase, wherein the substrate has a formula I 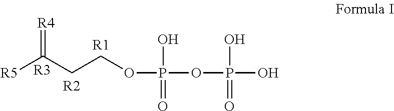
wherein R5=a hydroxy methyl group; an aniline or a styrene.

2. The composition of claim 1, further comprising a lipid capable of forming a higher order structure.

3. The composition of claim 2, wherein the higher order structure is a micelle, a liposome, a mono-layer membrane, or a bilayer membrane.

4. The composition of claim 2, wherein the lipid is DMPC.

5. The composition of claim 1, further comprising an isolated and purified small rubber particle protein.

6. The composition of claim 5, wherein the small rubber particle protein has a sequence at least 99% identical to SEQ ID NO: 10 or SEQ ID NO: 12.

7. The composition of claim 1, wherein the cis-prenyltransferase has a sequence at least 99% identical to SEQ ID NO: 2 or SEQ ID NO: 6

8. The composition of claim 1, wherein the cis-prenyltransferase binding protein has a sequence at least 99% identical to SEQ ID NO: 4 or SEQ ID NO: 8.

9. The composition of claim 1, further comprising a non-aqueous organic solvent.

10. The composition of claim 1, further comprising a divalent cation.

11. The composition of claim 1, wherein the divalent cation is Mg²⁺.

12. The composition of claim 1, wherein the initiator is an allylic pyrophosphate.

13. The composition of claim 1, wherein the allylic pyrophosphate is FPP.

14. A method of synthesizing rubber polymer, comprising:
a. providing reagents for synthesizing the rubber polymer;
b. providing the composition of claim 1; and
c. contacting the reagents with the composition of claim 1 under conditions allowing for the production of the rubber polymer, thereby synthesizing the rubber polymer.

15. The method of claim 14, wherein the composition of claim 1 further comprises a lipid capable of forming a higher order structure.

16. The method of claim 14, wherein the composition of claim 1 further comprises an isolated and purified small rubber particle protein.

17. The method of claim 14, wherein the composition of claim 1 further comprises an organic solvent.

18. The method of claim 14, wherein the composition in step b further comprises a divalent cation and wherein the divalent cation is optionally Mg²⁺.

19. A composition for producing rubber polymer in vitro, comprising:
a recombinant cis-prenyltransferase protein with a sequence at least 95% identical to a SEQ ID NO: 2 or SEQ ID NO: 6 incorporated into a membrane composed of lipid; a recombinant cis-prenyltransferase binding protein with a sequence at least 95% identical to SEQ ID NO: 4 or SEQ ID NO: 8 incorporated into a membrane composed of lipid; a reaction allylic pyrophosphate initiator; a substrate convertible into a polymer by said cis-prenyltransferase wherein the substrate has a formula I 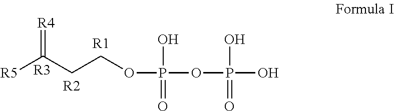
wherein R5=a hydroxy methyl group; an aniline or a styrene;
and an aqueous medium.


SEQUENCE LISTING (14 sequences)

SEQ ID NO: 1 - DNA, 915 bp, Parthenium argentatum
  source 1..915: mol_type = genomic DNA
atggaagtcaatccaatcatcacaacagataattccttgaaaaaaatggaagaagaaagatcagaaagtatgatgaccaagttcttaggaggcttaaatttaacagcaagaaagcttctttttcgggtcatttcaaccggcccgattccacaacatatagcattcatcatggatggaaaccgaagattcgccaagaaatggaagctgactgagggtggtgggcataaagccggattcttggcactcatgtcggtgctaaaatactgttatgaaataggtgtgaagtatgtgacggtttacgcattcagtcttgataacttcaataggcggccggatgaagttcagtatgtaatgaacttgatgcaagaaaagattgaagggtttttgaaagagttaacaattgtgaacaagtatggtgttagagtcttgtttattggtgatcttaaaaggttatacgagccggttaggattgcagccgagaaagcaatggaagccactgctcacaacacacatacatatcttttagtatgtgttgcttacacttcttcacatgagatcccacgtgctatttacgaagcttgtgaagagaagagtggtggaacccgagttatgatgatgaatgggagtgtgaatggagattacaataatggagattatgaggatcatggggagggggttaaagtggtggatattgataaacatatgtatatggcagtggctccggatcctgatattttggttaggagctccggggagacgagactgagcaactacttattgtggcaaaccactaattgtgtgttgtattcgccaaaagcgttgtggccggagatggggctgtggcaggtggtttgggggatcttgaagtttcagaaggattataagtatttggagaagaagaagaagcaggcttga

SEQ ID NO: 2 - protein, 304 aa, Parthenium argentatum
MEVNPIITTDNSLKKMEEERSESMMTKFLGGLNLTARKLLFRVISTGPIPQHIAFIMDGNRRFAKKWKLTEGGGHKAGFLALMSVLKYCYEIGVKYVTVYAFSLDNFNRRPDEVQYVMNLMQEKIEGFLKELTIVNKYGVRVLFIGDLKRLYEPVRIAAEKAMEATAHNTHTYLLVCVAYTSSHEIPRAIYEACEEKSGGTRVMMMNGSVNGDYNNGDYEDHGEGVKVVDIDKHMYMAVAPDPDILVRSSGETRLSNYLLWQTTNCVLYSPKALWPEMGLWQVVWGILKFQKDYKYLEKKKKQA

SEQ ID NO: 3 - DNA, 753 bp, Parthenium argentatum
  source 1..753: mol_type = genomic DNA
atggatctagtagctgaatcacagaagttttttcgcaggacctcgcagagtggcagcattgtgctcttcttgctctggcatgtagttcacttaacaatcagtgttttatacattgtccgggagatctttcgtgcgattgaaagctaccttataacaaacggatatgtgaaaacatacacaaatataaatttacaacgggtcaaatatcttggaattgttgtggacagtgatgaagcccgtaacatctcaaaagtggttgaacttttagagtggctttcagctataggtgtgaaaaagatctgtctttatgaccgggaaggagtgttgaagaagtcaaaggcggtcatcatggagagatttggctctacagagacttccaatgatagtgcagtagccaatccactaagtaaaaaacggatggattttgaatttgtttcaatcactgatggcaaagaagcagttgctaaagcagctaacctactctttaaaaaatattatgtggacgaagattcagaaaaaccattctttactgaaacccacttgaccgaggcactaaagaccctcgggcaagtagagccagatcccgatcttttattaatttacgggccagtgaggtgccaccttggttttccagcatggcgacttcgttacacggagatggtgcacatgggaccattaaagtacaagaaatttggtttgattctgaaagcaattcacaggttcactaaggtgaagcaaaactatggttcataa

SEQ ID NO: 4 - protein, 250 aa, Parthenium argentatum
MDLVAESQKFFRRTSQSGSIVLFLLWHVVHLTISVLYIVREIFRAIESYLITNGYVKTYTNINLQRVKYLGIVVDSDEARNISKVVELLEWLSAIGVKKICLYDREGVLKKSKAVIMERFGSTETSNDSAVANPLSKKRMDFEFVSITDGKEAVAKAANLLFKKYYVDEDSEKPFFTETHLTEALKTLGQVEPDPDLLLIYGPVRCHLGFPAWRLRYTEMVHMGPLKYKKFGLILKAIHRFTKVKQNYGS

SEQ ID NO: 5 - DNA, 1051 bp, Hevea brasiliensis
  source 1..1051: mol_type = genomic DNA
tggaggatttaccgagtcacctacaggcttcgggttaagtcagtggtttaaggaaaatggaattatacaacggtgagaggccaagtgtgttcagacttttagggaagtatatgagaaaagggttatatagcatcctaacccagggtcccatccctactcatattgccttcatattggatggaaacgggaggtttgctaagaagcataaactgccagaaggaggtggtcataaggctggatttttagctcttctgaacgtactaacttattgctatgagttaggagtgaaatatgcgactatctatgcctttagcatcgataattttcgaaggaaacctcatgaggttcagtacgtaatgaatctaatgctggagaagattgaagggatgatcatggaagaaagtatcatcaatgcatatgatatttgcgtgcgttttgttggtaatctgaagcttttagatgagccactcaagaccgcagcagataagataatgagggctactgccaaaaattccaaatttgtgcttctccttgctgtatgctacacttcaactgatgagatcgtgcatgctgttgaagaatcctctaaggataaattgaaatccgatgaaatttgcaacgatggaaacggagattgtgtgattaaaattgaggagatggagccatattctgaaataaaacttgtagagcttgagagaaacacttacataaatccttatcctgatgtcttgattcgaacttctggggagacccgtctgagcaactacctactttggcagactactaattgcatactgtattctcctcatgcactgtggccagagattggtcttcgacacgtggtgtgggcagtaattaactgccaacgtcattattcttacttggagaaacataaggaatacttaaaataatttgtttctgttcctagctcatcctgccttattcgcgatagttaagcttaagcatatccttgtagaataaacttggaaactcaagcaagggattaatccttgtattgtagcaaattccgacatgcaaaaaaaaaaaaaaa

SEQ ID NO: 6 - protein, 284 aa, Hevea brasiliensis
MELYNGERPSVFRLLGKYMRKGLYSILTQGPIPTHIAFILDGNGRFAKKHKLPEGGGHKAGFLALLNVLTYCYELGVKYATIYAFSIDNFRRKPHEVQYVMNLMLEKIEGMIMEESIINAYDICVRFVGNLKLLDEPLKTAADKIMRATAKNSKFVLLLAVCYTSTDEIVHAVEESSKDKLKSDEICNDGNGDCVIKIEEMEPYSEIKLVELERNTYINPYPDVLIRTSGETRLSNYLLWQTTNCILYSPHALWPEIGLRHVVWAVINCQRHYSYLEKHKEYLK

SEQ ID NO: 7 - DNA, 774 bp, Hevea brasiliensis
  source 1..774: mol_type = genomic DNA
atggatttgaaacctggagctggagggcagagagttaatcgtttagtggatccgattagttatcattttcttcaatttctgtggcgtactctacatcttcttgtcagcttatggtaccttcaagttagtatggtccaaatgatcgaaggctttctaatctctagtggacttgtgaaacgctatggagccctcgatattgacaaggtccggtaccttgccattgtggtagatagtgaagaagcttaccaaatttctaaagttattcagcttttgaaatgggtggaagatatgggtgtgaaacatttatgcctctatgattcaaaaggagttctcaagacaaacaagaaaaccatcatggagagtttgaacaatgctatgccatttgaggaagcagttgaaaaagatgttttactggaccagaaacagatgactgtggaatttgcttccagctccgatggaaaggaagcaataaccagggcagctaacgtactctttatgaagtatttgaagtatgctaaaactggtgtaggaaaggaagaaccatgctttacagaagatcaaatggatgaggcactaaaagctataggttacaaagggccggaacctgacttgctattaatttatggacctgttagatgccatctaggtttctcaccgtggagacttcgatatactgagatggtgcatatgggacccttgaggtacatgaacctcggttcactaaaaaaggccattcacaggttcacaacagtgcagcaaaattatggtacatga

SEQ ID NO: 8 - protein, 257 aa, Hevea brasiliensis
MDLKPGAGGQRVNRLVDPISYHFLQFLWRTLHLLVSLWYLQVSMVQMIEGFLISSGLVKRYGALDIDKVRYLAIVVDSEEAYQISKVIQLLKWVEDMGVKHLCLYDSKGVLKTNKKTIMESLNNAMPFEEAVEKDVLLDQKQMTVEFASSSDGKEAITRAANVLFMKYLKYAKTGVGKEEPCFTEDQMDEALKAIGYKGPEPDLLLIYGPVRCHLGFSPWRLRYTEMVHMGPLRYMNLGSLKKAIHRFTTVQQNYGT

SEQ ID NO: 9 - DNA, 912 bp, Parthenium argentatum
  source 1..912: mol_type = genomic DNA
ttcttctcctattccttattgtaatctccgtcactacaaacttctgttcttctagacgctcatggccgaacctgaatccaatcagtccgcccagcccgtggcagagagggatgggggagaggagcaactgcatctgaaatatcttgattttgtgcaaaacgctgtgatctattttgttgtttgtttctctactgtttacggttacgcaaaggagaacgccggttcgtggaagcctggtgttcaaaccgttgagaacaccgttctaaacgtcgttggaccggtttacgaaaagtattatgactatcctatagaggccctcaagttccttgatgtaaaggtgggcgacttggtgaccgagctgaaacggcacgtgccatcactaatgaaacaggcttcaagccaagccaaatacacggctcagaaccttccagaagtggctaaagccttggcaacagaggcattcaaaactgctacaaatgtggccaacacattgtacgtaaaatgcgagccaacagctaaacagctatacatgaactacgagccggtagctgagaaatacacggtgtcgacatggcggtcattgaacaaactccctttgtttcctcaggtagctcagattgcggttcctactggtgcttatgtgcttgagaagtataacgaccccgttagctacactgcggacaaaggttatgctgtggctcagtatttaccgttggttccgattgataaaattgctaaggtgtttaaaaagggtgagagcgggtcaacggttggtcaaagtggttaggtcgagacttgatgaattacgttggttccagtttataaatcaagtctatgataatggtttagttttaaaaaaaaaaaaaaaaaaaaaaaaaaaaaaaaaaaaaaaaaaaaaaaaaaaaaaaaaaa

SEQ ID NO: 10 - protein, 241 aa, Parthenium argentatum
MAEPESNQSAQPVAERDGGEEQLHLKYLDFVQNAVIYFVVCFSTVYGYAKENAGSWKPGVQTVENTVLNVVGPVYEKYYDYPIEALKFLDVKVGDLVTELKRHVPSLMKQASSQAKYTAQNLPEVAKALATEAFKTATNVANTLYVKCEPTAKQLYMNYEPVAEKYTVSTWRSLNKLPLFPQVAQIAVPTGAYVLEKYNDPVSYTADKGYAVAQYLPLVPIDKIAKVFKKGESGSTVGQSG

SEQ ID NO: 11 - DNA, 910 bp, Hevea brasiliensis
  source 1..910: mol_type = genomic DNA
cagtgttttccgaaaggcaaatcttttttcaaacttcagcgactgcgttttgaatttgtgatttttaaaggaaattttcaattatggctgaagaggtggaggaagagaggctaaagtatttggattttgtgcgagcggctggagtttatgctgtagattctttctcaactctctacctttatgccaaggacatatctggtccattaaaacctggtgtcgatactattgagaatgtggtgaagaccgtggttactcctgtttattatattccccttgaggctgtcaagtttgtagacaaaacggtggatgtatcggtcactagcctagatggcgttgttcccccagttatcaagcaggtgtctgcccaaacttactcggtagctcaagatgctccaagaattgttcttgatgtggcttcttcagttttcaacactggtgtgcaggaaggcgcaaaagctctgtacgctaatcttgaaccaaaagctgagcaatatgcggtcattacctggcgtgccctcaataagctgccactagttcctcaagtggcaaatgtagttgtgccaaccgctgtttatttctctgaaaagtacaacgatgttgttcgtggcactactgagcagggatatagagtgtcctcttatttgcctttgttgcccactgagaaaattactaaggtgtttggagatgaggcatcataatctgcactggatttggttatttatctattgtgagcttttttatatgtacttattcagtgtttagaataagtctttggtggtgtgttttggatgtggaataaagggccaattgcattgttggtcaatatataattatgtataacatttcgtgatttgagttggaatctaaaggttttattaagaatggatggactaaaaaaaaaaaaaaaaaa

SEQ ID NO: 12 - protein, 204 aa, Hevea brasiliensis
MAEEVEEERLKYLDFVRAAGVYAVDSFSTLYLYAKDISGPLKPGVDTIENVVKTVVTPVYYIPLEAVKFVDKTVDVSVTSLDGVVPPVIKQVSAQTYSVAQDAPRIVLDVASSVFNTGVQEGAKALYANLEPKAEQYAVITWRALNKLPLVPQVANVVVPTAVYFSEKYNDVVRGTTEQGYRVSSYLPLLPTEKITKVFGDEAS

SEQ ID NO: 13 - DNA, 681 bp, Hevea brasiliensis
  source 1..681: mol_type = genomic DNA
ttttgatcttccatttttgcaaaaggaaatcttcgattatggctgaagacgaagacaaccaacaagggcagggggaggggttaaaatatttgggttttgtgcaagacgcggcaacttatgctgtgactaccttctcaaacgtctatctttttgccaaagacaaatctggtccactgcagcctggtgtcgatatcattgagggtccggtgaagaacgtggctgtacctctctataataggttcagttatattcccaatggagctctcaagtttgtagacagcacggttgttgcatctgtcactattatagatcgctctcttcccccaattgtcaaggacgcatctatccaagttgtttcagcaattcgagctgccccagaagctgctcgttctctggcttcttctttgcctgggcagaccaagatacttgctaaggtgttttatggagagaattgagccccaatttgcaccaattgcttccaactaagcaagttaatgatatgctcaagaatatatatctattgtgagctttttttatgttctcatcctgagtgttgagactatgttttcgtttgaatattatactgtgttttattatgtgttttgaatattcataatgagaataaagggccaattgaattattggccaatatgtaatgatacataaatttcgtgattgagt

SEQ ID NO: 14 - protein, 138 aa, Hevea brasiliensis
MAEDEDNQQGQGEGLKYLGFVQDAATYAVTTFSNVYLFAKDKSGPLQPGVDIIEGPVKNVAVPLYNRFSYIPNGALKFVDSTVVASVTIIDRSLPPIVKDASIQVVSAIRAAPEAARSLASSLPGQTKILAKVFYGEN